(19) United States
(12) Patent Application Publication
(10) Pub. No.: US 2025/0248863 A1
(43) Pub. Date: Aug. 7, 2025

(54) PERSONAL NASAL AND FACIAL EXCRETION ABSORBER APPARATUSES

(71) Applicant: Catherine Majkowski, West Bend, WI (US)

(72) Inventor: Catherine Majkowski, West Bend, WI (US)

(21) Appl. No.: 19/042,655

(22) Filed: Jan. 31, 2025

Related U.S. Application Data
(60) Provisional application No. 63/548,666, filed on Feb. 1, 2024.

(51) Int. Cl. A61F 13/45

(52) U.S. Cl. CPC A61F 13/45

(57) ABSTRACT

An apparatus for wiping fluids from a user includes a sleeve configured to be worn by the user, a compartment defining an interior space, a wiping member coupled to the sleeve and being moveable into and between a first position in which the wiping member is in the interior space and a second position in which the wiping member extends from the interior space such that the user can use the wiping member to wipe fluids.

### CROSS-REFERENCE TO RELATED APPLICATION

The present disclosure is based on and claims priority to U.S. Provisional Patent Application No. 63/548,666, filed Feb. 1, 2024, the disclosure of which is incorporated herein by reference.

### FIELD

The present disclosure relates to apparatuses for absorbing personal nasal and facial excretions.

### BACKGROUND

Humans excrete fluids from their eyes, nose, mouth, and skin, and these fluids often must be removed for comfort, physical safety, and/or to prevent spreading the fluids to other people.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In certain examples, an apparatus for wiping fluids from a user includes a sleeve configured to be worn by the user, a compartment defining an interior space, a wiping member coupled to the sleeve and being moveable into and between a first position in which the wiping member is in the interior space and a second position in which the wiping member extends from the interior space such that the user can use the wiping member to wipe fluids.

In certain examples, an apparatus for wiping fluids from a user includes a sleeve configured to be worn by the user, a compartment defining an interior space, the compartment having a compartment width and a compartment length, a wiping member coupled to the sleeve and being moveable into and between a first position in which the wiping member is stored in the interior space and a second position in which the wiping member extends from the interior space such that the user can use wiping member to wipe fluids, the wiping member has a wiping member width and a wiping member length, the wiping member length is greater than the compartment length.

Various other features, objects, and advantages will be made apparent from the following description taken together with the drawings.

### BRIEF DESCRIPTION OF THE DRAWINGS

The present disclosure is described with reference to the following Figures. The same numbers are used throughout the Figures to reference like features and like components.

FIG. 1 is a perspective view of an example apparatus according to the present disclosure.

FIG. 2 is a perspective view of the example apparatus coupled to a body part of a user.

FIG. 3 is a side view of another example apparatus according to the present disclosure with a wiping member in an interior space.

FIG. 4 is a side view of another example apparatus according to the present disclosure with a wiping member in an interior space.

FIG. 5 is a view like FIG. 4 with the wiping member removed from the interior space.

FIG. 6 is a side view of another example apparatus according to the present disclosure.

FIG. 7 is a perspective view of another example apparatus according to the present disclosure.

FIG. 8 is a perspective view of another example apparatus according to the present disclosure.

### DETAILED DESCRIPTION

The present inventor recognized that humans excrete fluids from their eyes, nose, mouth, and skin during activities such as walking, running, biking, hiking, and the like. The person excreting the fluids often wishes to remove the fluids to minimize discomfort and/or reduce the spread of germs. Conventional face tissues may be used, but the person must pack the tissues before commencing the activity. The person may also try to eject the fluid by shaking or blowing the fluids off (e.g., launching snot out of one's nose and through the air commonly known as a “Farmer Blow” or “Snot Rocket”). These options are unsanitary, unpredictable, and not desirable.

The present inventor further recognized that exercise induced rhinitis (EIR), generally described as runny nose during exercise activities has been occurring for one hundred plus years to athletes and active people. Some studies estimate that between 25.0-50.0% of athletes describe experiencing a runny nose during exercise and EIR. In certain examples, while nasal drainage increases (i.e. runny nose), nasal congestion/blockage will typically decrease, and some studies have shown that during exercise, the resistance of our nasal passages can decrease, and they become more open to allow more air to pass through. A runny nose during exercise and/or EIR are natural, physical body occurrences that increase nasal drainage and can occur in all manner of sports and physical activities including but not limited to common land/outdoor activities and every day activities such as biking, running, hiking, mountain climbing, walking, weight lifting, yoga, martial arts, sportsman hunting, fishing, first responder and military training, skiing & snowboarding, ice skating, gardening, and planting. The exact mechanisms for EIR are unclear, but it appears that EIR is likely related to circumstance, activity, environment, and individual health. In addition, a runny nose can develop during more simple and common circumstances such as the cold air causing a runny nose. A runny nose can occur when the nerves are activated in the nose, stimulating the lining of the nose to produce more mucus, causing “vasomotor rhinitis”. Also referred to as “skier's nose”, which has been researched and is common in at least 50.0% of athletes (or the average Joe) who play winter sports.

Additional studies have also shown that EIR occurs more frequently in people who suffer from allergies (AR-allergic rhinitis). It is possible that in some people, the increased airflow passing through the nose when exercising (nasal decongestion and increased respiration/breathing) can introduce more air-born allergens in the nose, contributing to a runny nose. This can occur outside in nature or in an inside studio, gym, or home exercise environment. Allergens, chemicals, and cleaning products also act as irritants to the lining of the nose. All of which are present and increasing in our environments today, both inside and outdoors.

There is no known cure for EIR, but the present inventor endeavored to develop solutions that are much wiser, earth friendly, and economical to account for the occurrence of this annoying thing that happens naturally. Allergy medicine and nasal sprays may work temporarily for those with allergies or sensitivities, offering intermittent, temporary relief, but it does not take away the reality of EIR. The nose runs, eyes water, and people sweat, especially when exerting energy during exercise, training, work, or play. Tears are a clear liquid secreted by the lacrimal glands (tear glands) found in our eyes. Tears also naturally drain into openings, called puncta, on the inside corners of your upper and lower eyelids. The puncta lead to small canals called canaliculi. Canaliculi move tears to a sac to a reservoir on the side of the nose called the lacrimal sac. From there, tears travel down the nasolacrimal duct and drain into the nose. This natural process also contributes to a runny nose, so we now have tears and snot running down the face. This all needs to be wiped.

The present inventor also recognized that conventional face tissues can be costly and become a waste product set to landfills. Studies have shown that Americans use billions of disposable facial tissues each year. In one study, 20.0% respondents said they use five to seven tissues daily, about 10.0% percent of respondents admitted to using eight to ten tissues per day, 3.2% percent of respondents used 11-15 tissues per day, and 3.0% percent of respondents used more than 15 tissues per day. When at home, 40.0% percent of respondents said they used two to four tissues a day. This does not include the potential number of tissues an active person would use, when engaged in sports or movement when EIR is present. See for example article “Exercise-induced rhinitis: why does my nose run when I exercise?” by Ahmad R. Sedaghat, MD, PHd, FACS Feb. 13, 2021.

Handkerchiefs are still used, but appear to be most common with older generations. Millennials and Gen Z'ers are switching to handkerchiefs, and it's not just to make a statement it is because handkerchiefs can be practical, convenient, eco-friendly, recyclable, and/or washable. Disposable tissues can negatively impact the environment when left on trails, in parks, on the streets, and/or in training facilities when the garbage cans are over-flowing, such that the tissues are left to blow in the wind and add additional trash and pollution to our outdoor environments. Tissues also take a long time to break down in the environment. In the National Parks, you must take out what you carry in. Mountain climbers, hikers, campers, and those who spend a lot of time outdoors, know the importance of using recyclable items as often as possible to lighten your load and be eco-friendly to the delicate environment. For those engaging in active athletic competitions, events, or action-focused activities it will be much safer to wipe your snot, sweat, and tears on clothing attached to your person, rather than searching and digging for tissues or a handkerchief in a pocket.

Through research and experimentation, the present inventor developed the example apparatuses 10 of the present disclosure that facilitate removal of undesirable fluids from the person thereby increasing the comfort of the person during continued activity. The example apparatuses 10 of the present disclosure can be utilized by enthusiasts/active lifestyle participants, hobbyists, fitness-minded individuals, and/or workers. The example apparatuses 10 of the present disclosure reduce or eliminate the need for the person to shake or blow undesirable fluids from the person and/or provide the person with an alternative to wiping the fluids on their clothes. In addition, the example apparatuses 10 of the present disclosure are attached to the body of the person thereby eliminating the need to carry separate articles (e.g., handkerchiefs, tissues). The example apparatuses 10 of the present disclosure reduce disposable articles (e.g., paper tissues that are sent to the landfill) and are thus an earth-friendly, eco-friendly solutions to a real problem. In certain examples, the apparatuses 10 of the present disclosure can be utilized by mothers or fathers who need to wipe the face of a baby when out for a walk, kids as an alternative to tissues in school during cold and flu season, elderly in senior communities, and/or special needs children.

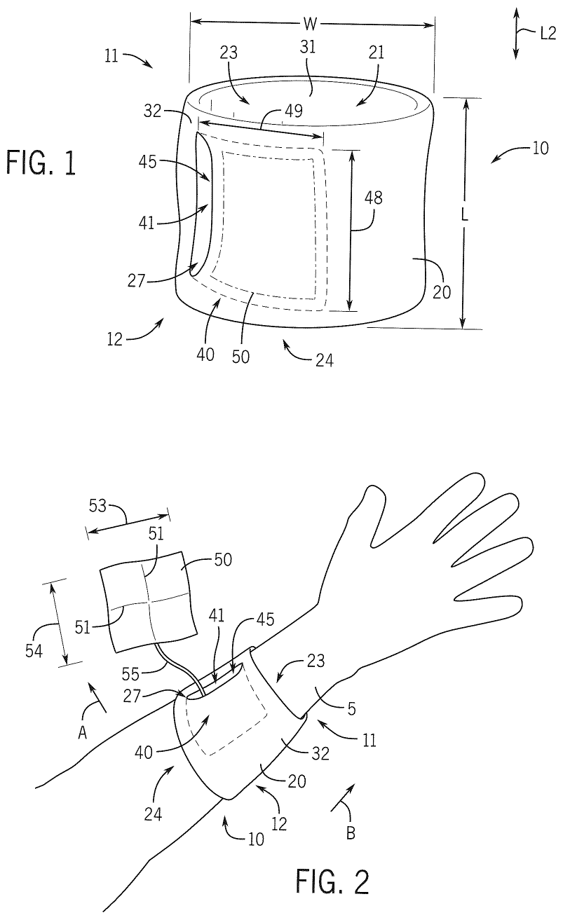

FIGS. 1-2 depicts an example apparatus 10 according to the present disclosure, and the apparatus 10 is configured to be worn by a user (as depicted in FIG. 2). The user can easily access the apparatus 1 when a fluid needs to be removed and/or absorbed away from the body.

The apparatus 10 generally extends longitudinally between a first end 11 and an opposite second end 12 (see example longitudinal axis L2), and the apparatus 10 has a sleeve 20 that generally encircles a center void 21. The sleeve 20 further defines a first opening 23 and a second opening 24 into the void 21.

The sleeve 20 includes an inner surface 31 that is configured to face the skin of user, such as the skin of a body part, such as a forearm 5. An opposite outer surface 32 faces away from the forearm 5. The textures of the surfaces 31, 32, in some examples, are the same. For instance, the surfaces 31, 32 both have the same textures (e.g., soft, absorbent, stretchy). In other examples, the textures of the surfaces 31, 32 are different. For example, the inner surface 31 is ribbed while the outer surface 32 is plush. Note that in other examples the apparatus 10 can be worn on other parts of the body including but not limited to the wrist, bicep area of the arm, or an elbow.

The length L of the sleeve can vary, and in one example, the length L is 5.0 inches (″) The width W of the sleeve can also vary, and in one example, the width W is 4.0″. The sleeve 20 can made into different sizes such as small/medium or large/extra-large to match the size of the user. In one example, the sleeve 20 has a width of 4.0″ and a length of 5.0″. In another example, the sleeve 20 has a width of 5.0″ and a length of 6.0″.

The apparatus 10 includes a pocket or compartment 40 having an interior space 41 (described further herein). In one example, an additional piece of fabric or other material (e.g., plastic film, canvas) is attached to the outer surface 32 of the sleeve 20 such that the compartment 40 is defined between the outer surface 32 and the additional piece of material. In this example, the interior space 41 is between the outer surface 32 and the additional pieces of material. In other examples, such as the example depicted in FIGS. 1-2, the compartment 40 is in the sleeve and the interior space 41 is defined between two layers of material that form the sleeve 20 (e.g., two layers of material forming the sleeve 20 with one layer having the outer surface 32 and one layer having the inner surface 31). A compartment opening 45 allows for passage into and out of the interior space 41 of the compartment 40. In certain examples, the compartment opening 45 is defined by a slit or slot 27 in the sleeve 20. In these examples, layers of the sleeve 20 may define the extents of the interior space 41. The compartment 45 has a compartment width 48 and a compartment length 49.

In the example depicted in FIGS. 1-2, the compartment opening 45 faces or is oriented in a second direction (arrow A) while the first opening 23 faces or is oriented in a first direction (arrow B).

In certain examples, the compartment 40 has a liner (not depicted) that is a separate additional pieces of fabric or material that defines the extents of the interior space 41 and the liner can be coupled to the sleeve 20 adjacent to the slot 27. The liner could also be sandwiched between layers of the sleeve 20 or sandwiched between the skin of the user and the inner surface 31.

The apparatus 10 includes a wiping member 50 (depicted in dash-dot lines in FIG. 1) that is configured to be stored in the compartment 40 and can be selectively removed from the compartment 40 (see FIG. 2) by the user when the user desires to wipe fluid away. For example, the wiping member 50 is moveable into and between a first position (FIG. 1) in which the wiping member 50 is in the interior space 41 and a second position (FIG. 2) in which the wiping member is removed and/or extends out of the interior space 41. In the first position, the wiping member 50 is stored for future use by the user, and in the second position the wiping member 50 can be used by user to wipe fluids of the user.

The wiping member 50 can be formed of any suitable material (described further therein). The wiping member 50 can be folded (see example fold lines 51 in FIG. 2). The wiping member 50 has an unfolded wiping member width 53 and an unfolded wiping member length 54. In certain examples, the wiping member width 53 is greater than the compartment width 48 and/or the member length 54 is greater than the compartment length 49 such that the wiping member 50 is folded before being inserted into the interior space 41 of the compartment 40. In certain examples, the wiping member 50 has a length of 15.0-18.0 inches. In certain examples, the wiping member width 53 corresponds to the compartment width 48.

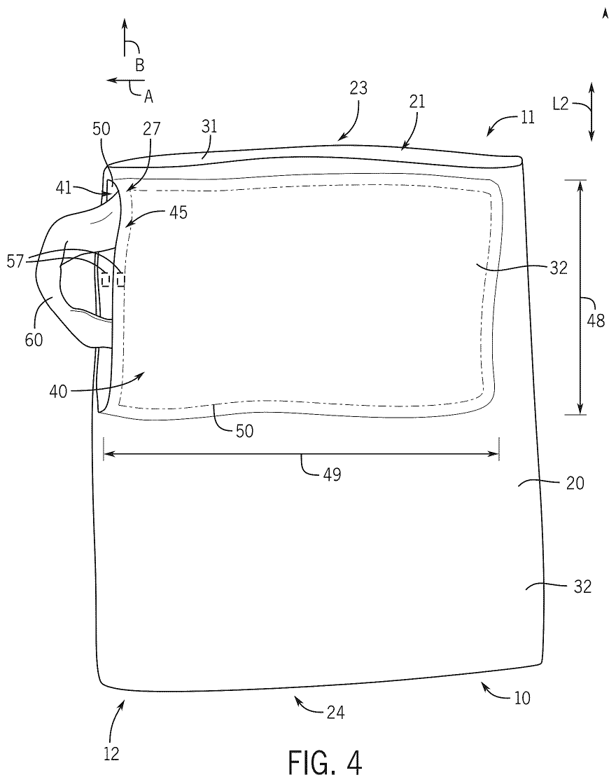

In certain examples, a closure device 57 (see FIG. 4) closes the compartment opening 51 to thereby prevent inadvertent movement of the wiping member 50 out of the interior space 41. The closure device 57 can vary, and some example closure devices 50 include but are not limited to hook-and-loop strips (e.g., Velcro strips), buttons, snaps, and stretch cording or piping.

In certain examples, a leash 55 couples the wiping member 50 to the sleeve 20. The leash 55 prevents the wiping member 50 from being inadvertently disconnected from the apparatus 10. When the wiping member 50 is stored, the leash 55 may be positioned in the compartment 40 or along the outer surface 32 of the sleeve 20. The leash 55 can be formed of elastic material (e.g., spandex, rubber) such that the leash 55 stretches as the user pulls the wiping member 50 away from the sleeve 20. In certain examples, the leash 55 includes hook-and-loop fasteners for coupling the leash 55 to the sleeve 20.

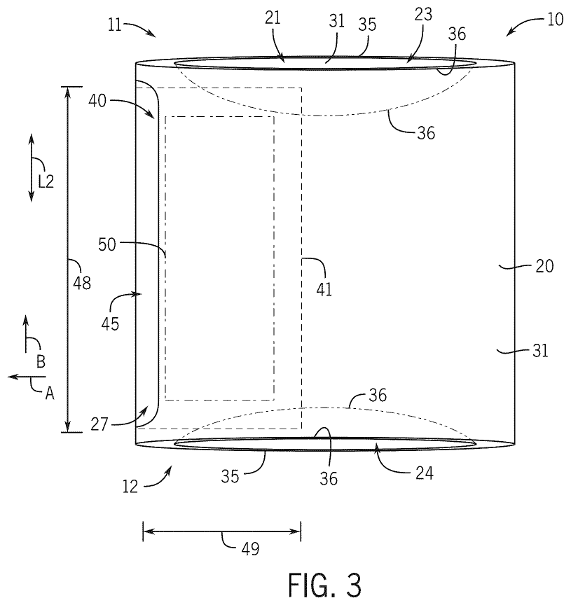

FIG. 3 depicts another example apparatus 10 according to the present disclosure. The example apparatuses 10 described further herein, such as the example depicted in FIG. 3, can include any of the features and/or components (e.g., the first end and the second end of the apparatus 10, the leash 55) noted above with respect to the example apparatus 10 depicted in FIGS. 1-2. Similarly, the example apparatuses 10 described above, such as the example apparatus depicted in FIGS. 1-2, can include any of the features and/or components of the example apparatuses described below. Note that the features and/or components of the example apparatuses described in this disclosure that are similar are denoted with the same part numbers, however, the use of the same part numbers for features and/or components of various example apparatuses of the present disclosure should not be construed to indicate that the similarly marked features and/or components are necessarily identical. Instead, the similarly marked features and/or components may have varying characteristics. For example, the sleeve 20 depicted in FIGS. 1-2 can have a greater length than the sleeve 20 depicted in FIG. 3 and the wiping member 50 of FIGS. 1-2 has a different shape than the shape of the wiping member 50 depicted in FIG. 4.

The example apparatus 10 depicted in FIG. 3 includes the openings 23, 24, and edges 35, 36 surround the openings 23, 24. The edges include a first edge 35 and a second edge 36. In certain examples, the second edge 36 is curved (see dash-double-dot lines 36 in FIG. 3) such that the curved edge 36 of each opening 23, 24 longitudinally extends toward each other. The interior space 41 is depicted with dash lines in FIG. 4 and the wiping member 50 (depicted in dash-dot lines) is in the interior space 41. The compartment 40 has a compartment width 48 and a compartment length 49. Note that the dimensions of the compartment 40 depicted in FIG. 3 is different from the dimensions of the compartment 40 depicted in FIGS. 1-2. The dimensions, size, and/or shape of the compartment 40 can vary.

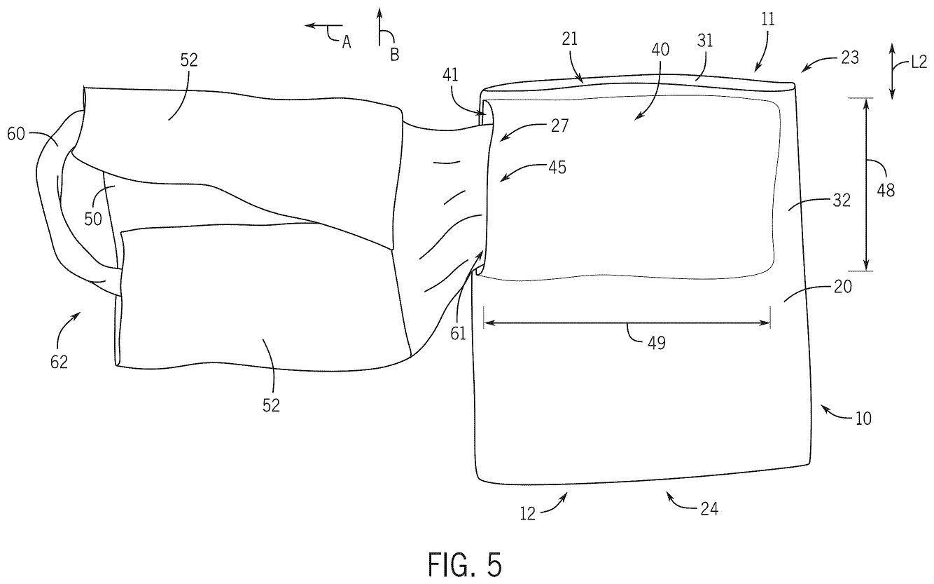

FIG. 4-5 depicts another example apparatus 10 according to the present disclosure. The example apparatus 10 depicted in FIGS. 4-5 can include any of the features and/or the components noted above with respect to the example apparatuses 10 depicted in FIGS. 1-2 and FIG. 3, and similarly, the example apparatus 10 depicted in FIGS. 1-2 and FIG. 3 can include any of the features and/or the components noted below with respect to the example apparatus 10 depicted in FIGS. 4-5.

FIG. 4 depicts the wiping member 50 (see dash-dot lines) in the compartment 40, and a handle 60 attached to the wiping member 50 extends out from the interior space 41 and away from the sleeve 20. Note that the handle 60 can be formed of the same material that forms the wiping member 50 or a different material. The handle 60 permits the user of the apparatus 10 to quickly and easily pull out the wiping member 50 out of the compartment 40 (see FIG. 5). In certain examples, the handle 60 extends out of the interior space 41 when the wiping member 50 is in the interior space 41. FIG. 5 depicts the wiping member 50 that was directed in FIG. 4 in the compartment 40 as pulled out of the compartment 40. The wiping member 50 has one or more sections 52 which are folded onto each other (as depicted in FIG. 5). The sections 52 are folded on to each other when the wiping member 50 inserted back into the compartment 40 and unfolded when the wiping member 50 is pulled out of the compartment 40 to increase the surface area of the wiping member 50 available to the user.

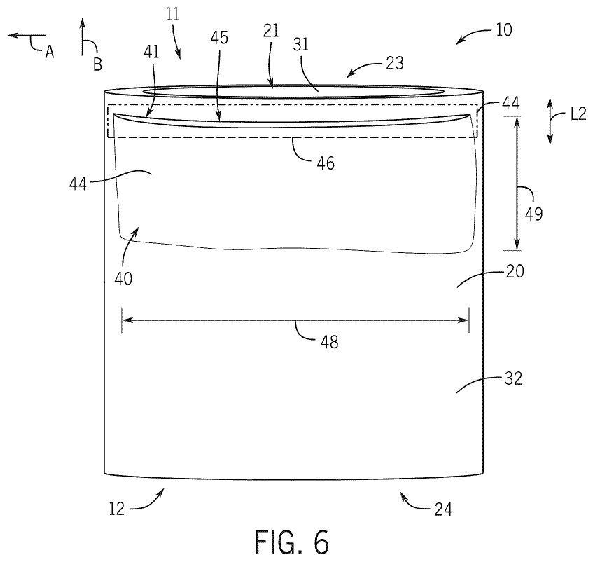

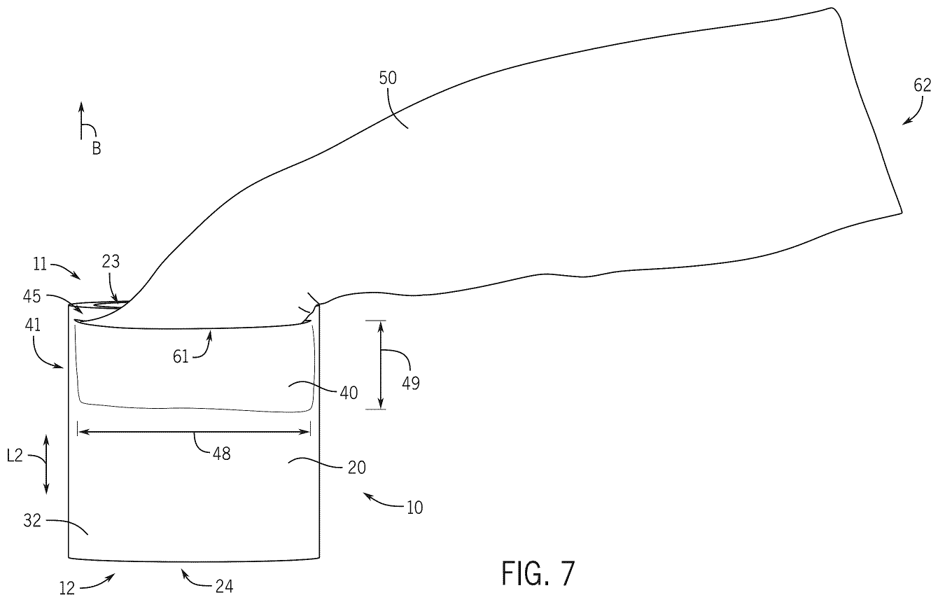

FIG. 6 depicts another example apparatus 10 according to the present disclosure. The example apparatus 10 depicted in FIG. 6 can include any of the features and/or the components noted above with respect to the example apparatuses 10 depicted in FIGS. 1-2, FIG. 3, and FIG. 4-5, and similarly, the example apparatus 10 depicted in FIGS. 1-2, FIG. 3, and FIG. 4-5, can include any of the features and/or the components noted below with respect to the example apparatus 10 depicted in FIG. 6.

In the example depicted in FIG. 6, the compartment opening 45 is oriented toward the opening 23 into the sleeve 20. For example, the compartment opening 45 extends along the first opening 23. The wiping member 50 can then be removed from the compartment 40 by pulling in the first direction (arrow B) toward the first opening 23. The apparatus 10 includes a flap 44 (double-dot-dash line) that is moveable about a hem line 46 (dashed line) to cover or expose the compartment opening 45 and/or the wiping member 50 in the interior space 41.

FIG. 7 depicts another example apparatus 10 according to the present disclosure. The example apparatus 10 depicted in FIG. 7 can include any of the features and/or the components noted above with respect to the example apparatuses 10 depicted in FIGS. 1-2, FIG. 3, FIGS. 4-5, and FIG. 6, and similarly, the example apparatuses 10 depicted in FIGS. 1-2, FIG. 3, FIGS. 4-5, and FIG. 6, can include any of the features and/or the components noted below with respect to the apparatus 10 depicted in FIG. 7.

The apparatus 7 depicted in FIG. 7 includes the compartment opening 45 that is oriented toward the opening 23 into the sleeve 20. The wiping member 50 is depicted as extended out from the interior space 41. The wiping member 50 includes a first member end 61 that is coupled to the sleeve 20 at a location in or near the compartment 40. The wiping member 50 includes a free second member end 62. In one example, the first member end 61 is coupled (e.g., sewn) to the sleeve 20. Note that the first member end 61 remains coupled to the sleeve 20 when the second member end 62 is extended from the compartment 40 by the user. By coupling the first member end 61 to the sleeve 20, the wiping member 50 does not inadvertnetly fall from the apparatus 10 onto the ground. In certain examples, the wiping member 50 is rolled up such that the wiping member 50 is easily inserted back into the interior space 41. In certain examples, the second member end 62 includes a handle (not depicted in FIG. 7, see example handle 60 in FIG. 4) on the second member end 62 to facilitate removal of the wiping member 50 from the interior space 41.

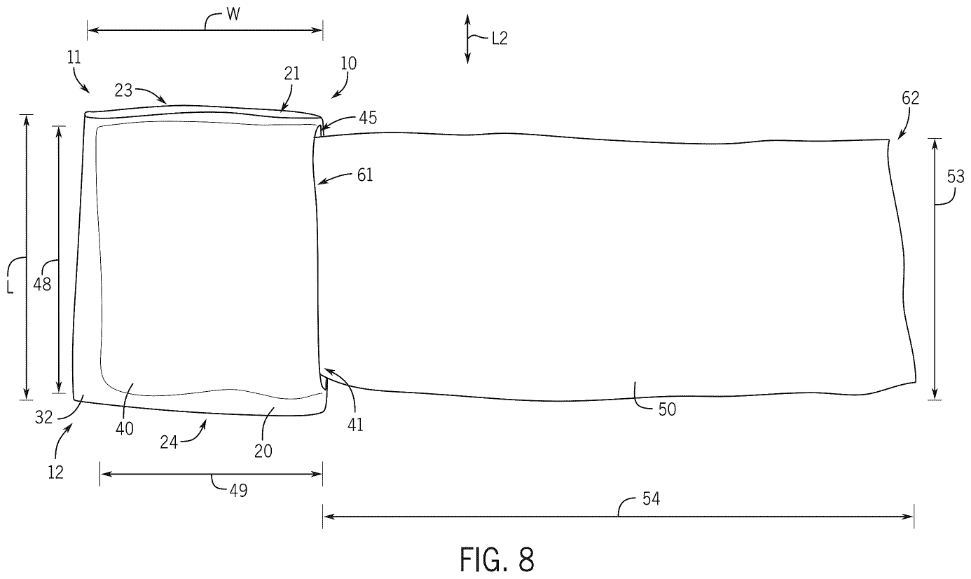

FIG. 8 depicts another example apparatus 10 according to the present disclosure. The example apparatus 10 depicted in FIG. 8 can include any of the features and/or the components noted above with respect to the example apparatuses 10 depicted in FIGS. 1-2, FIG. 3, FIGS. 4-5, FIG. 6, and FIG. 7, and similarly, the example apparatuses 10 depicted in FIGS. 1-2, FIG. 3, FIGS. 4-5, FIG. 6, and FIG. 7 can include any of the features and/or the components noted below with respect to the example apparatus 10 depicted in FIG. 8.

The apparatus 7 depicted in FIG. 8 includes the compartment opening 45 that is oriented in the second direction (arrow A). The wiping member 50 is depicted as extended out from the interior space 41 of the compartment 40. The wiping member 50 includes a first member end 61 that is coupled to the sleeve 20 at a location in or near the compartment 40. The wiping member 50 includes a free second member end 62. In one example, the first member end 61 is coupled (e.g., sewn) to the sleeve 20 at a location within the interior space 41 and/or to the sleeve 40. In this example, the interior space 41 is located between two layers of fabric and lines denoted as compartment 40 in FIG. 4 represent the extents of the compartment 40 and the area of the outermost layer of fabric of the sleeve 20 that bulges outwardly when the wiping member 50 is inserted and stored into the interior space 41.

The example apparatuses 10 of the present disclosure can be formed of any suitable materials. For example, the sleeve 20 is formed with elastic material such that the sleeve 20 stretches as the user pulls the apparatus 10 onto their body. The sleeve 20 that elastically ‘shrinks’ and applies a slight compression force on the body part that keeps the sleeve 20 on the user. The sleeve 20 can be formed of nylon, a cotton nylon blend, and/or the like.

The sleeve 20 is formed on flexible material such that the sleeve 20 can collapse onto itself (see FIG. 3) or folded when the sleeve 20 is not on the body part. In certain examples, the sleeve 20 is formed on a single layer of material. In other examples, the sleeve 20 is formed with two layers of materials which are coupled together and can be reversible for added use. In certain examples, the material forming the sleeve 20 and/or the wiping member 50 will incorporate dri-fit technology, drawing excretions of sweat, snot, and tears from the body. The material forming the sleeve 20 and/or the wiping member 50 can be a soft, skin-friendly texture, and dispersing the secretions across the fabric allowing the moisture to evaporate faster thereby giving a “dry feeling” to the sleeve 20 and/or the wiping member 50. In certain examples, the material forming the sleeve 20 and/or the wiping member 50 will incorporate a micro-fiber/polyester blend with nylon or spandex (elastane) for elasticity. For instance, material comprising 80.0% nylon and 20.0% spandex; 95.0% polyester and 5.0% spandex (elastane); or 88.0% polyester and 12.0% spandex. The material forming the sleeve 20 and/or the wiping member 50 can be designed to “wick away” moisture and dry faster in comparison to cotton fabrics. In certain examples, the material forming the sleeve 20 and/or the wiping member 50 is polyester. In certain examples, antibacterial compounds are woven into the material forming the sleeve 20 and/or the wiping member 50 to increase mildew and germ resistance. The material forming the sleeve 20 and/or the wiping member 50 can be easily washable, shrink resistant and dries quickly.

In certain examples, an apparatus for wiping fluids from a user includes a sleeve configured to be worn by the user, a compartment defining an interior space, a wiping member coupled to the sleeve and being moveable into and between a first position in which the wiping member is in the interior space and a second position in which the wiping member extends from the interior space such that the user can use the wiping member to wipe fluids.

Optionally, the wiping member has a first member end coupled to the sleeve and a free second member end. Optionally, a handle is coupled to the second member end and when the wiping member is in the interior space, the handle extends out of the interior space. Optionally, the compartment has a compartment width and the wiping member has a wiping member width that is greater than the compartment width. Optionally, the sleeve has a first opening and a second opening through which the user inserts a body part to wear the sleeve, the first opening faces in a first direction and the compartment has a compartment opening that faces in a second direction. Optionally, the sleeve has a first opening and a second opening through which the user inserts a body part to wear the sleeve, the first opening faces in a first direction and the compartment has a compartment opening that faces in the first direction. Optionally, a handle coupled to the wiping member and extending out of the interior space when the wiping member is in the interior space. Optionally, a flap selectively covers a compartment opening. Optionally, the sleeve includes elastic material such as the sleeve is configured to compress a body part of the user when the sleeve is worn by the user. Optionally, the compartment has a compartment opening into the interior space and a closure device that selectively closes the compartment opening to prevent inadvertent movement of the wiping member out of the interior space. Optionally, the closure device is a hook-and-loop strip. Optionally, the sleeve is formed with two layers of materials. Optionally, a leash couples the wiping member to the sleeve. Optionally, the leash includes elastic material. Optionally, the wiping member has a first member end coupled to the leash and a free second member end and a handle coupled to the second member end.

In certain examples, an apparatus for wiping fluids from a user includes a sleeve configured to be worn by the user, a compartment defining an interior space, the compartment having a compartment width and a compartment length, a wiping member coupled to the sleeve and being moveable into and between a first position in which the wiping member is stored in the interior space and a second position in which the wiping member extends from the interior space such that the user can use wiping member to wipe fluids, the wiping member has a wiping member width and a wiping member length, the wiping member width is greater than the compartment width. Optionally, the wiping member length is greater than the compartment length.

In the present description, certain terms have been used for brevity, clarity, and understanding. No unnecessary limitations are to be inferred therefrom beyond the requirement of the prior art because such terms are used for descriptive purposes and are intended to be broadly construed. The different apparatuses, systems, and method steps described herein may be used alone or in combination with other apparatuses, systems, and methods. It is to be expected that various equivalents, alternatives, and modifications are possible within the scope of the appended claims.

This written description uses examples to disclose the invention and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An apparatus for wiping fluids from a user, the apparatus comprising:
a sleeve configured to be worn by the user;
a compartment defining an interior space; and
a wiping member coupled to the sleeve and being moveable into and between:
a first position in which the wiping member is in the interior space; and
a second position in which the wiping member extends from the interior space such that the user can use the wiping member to wipe fluids.

2. The apparatus according to claim 1, wherein the wiping member has a first member end coupled to the sleeve and a free second member end.

3. The apparatus according to claim 2, further comprising a handle coupled to the second member end, and wherein when the wiping member is in the interior space, the handle extends out of the interior space.

4. The apparatus according to claim 1, wherein the compartment has a compartment width, and wherein the wiping member has a wiping member width that is greater than the compartment width.

5. The apparatus according to claim 1, wherein the sleeve has a first opening and a second opening through which the user inserts a body part to wear the sleeve, the first opening faces in a first direction and wherein the compartment has a compartment opening that faces in a second direction.

6. The apparatus according to claim 1, wherein the sleeve has a first opening and a second opening through which the user inserts a body part to wear the sleeve, the first opening faces in a first direction and wherein the compartment has a compartment opening that faces in the first direction.

7. The apparatus according to claim 1, further comprising a handle coupled to the wiping member and extending out of the interior space when the wiping member is in the interior space.

8. The apparatus according to claim 1, further comprising a flap that selectively covers a compartment opening.

9. The apparatus according to claim 1, wherein the sleeve includes elastic material such as the sleeve is configured to compress a body part of the user when the sleeve is worn by the user.

10. The apparatus according to claim 1, wherein the compartment has a compartment opening into the interior space, and further comprising a closure device that selectively closes the compartment opening to prevent inadvertent movement of the wiping member out of the interior space.

11. The apparatus according to claim 10, wherein the closure device is a hook-and-loop strip.

12. The apparatus according to claim 1, wherein the sleeve is formed with two layers of materials.

13. The apparatus according to claim 1, further comprising a leash that couples the wiping member to the sleeve.

14. The apparatus according to claim 13, wherein the leash includes elastic material.

15. The apparatus according to claim 13, wherein the wiping member has a first member end coupled to the leash and a free second member end, and further comprising a handle coupled to the second member end.

16. An apparatus for wiping fluids from a user, the apparatus comprising:
a sleeve configured to be worn by the user;
a compartment defining an interior space, the compartment having a compartment width and a compartment length; and
a wiping member coupled to the sleeve and being moveable into and between:
a first position in which the wiping member is stored in the interior space; and
a second position in which the wiping member extends from the interior space such that the user can use wiping member to wipe fluids;
wherein the wiping member has a wiping member width and a wiping member length;
wherein the wiping member length is greater than the compartment length.

17. The wiping member according to claim 16, wherein the wiping member width is greater than the compartment width.
